(19) United States
(12) Patent Application Publication
(10) Pub. No.: US 2021/0138463 A1
(43) Pub. Date: May 13, 2021

(54) METHODS FOR REDUCING BUBBLE FORMATION DURING INCUBATION

(71) Applicant: Centrillion Technologies, Inc., Palo Alto, CA (US)

(72) Inventors: Filip CRNOGORAC, Redwood City, CA (US); Eric KIM, Sunnyvale, CA (US); Uzair SIKORA, San Jose, CA (US)

(21) Appl. No.: 17/071,988

(22) Filed: Oct. 15, 2020

Related U.S. Application Data
(60) Provisional application No. 62/915,079, filed on Oct. 15, 2019.

(51) Int. Cl. B01L 3/00; B01L 7/00; C12Q 1/6813

(52) U.S. Cl. CPC B01L 3/502715; B01L 7/52; B01L 2400/0409; B01L 2300/0829; C12Q 1/6813

(57) ABSTRACT

The present disclosure provides methods and apparatus for reducing bubble formation during incubation processes. The incubation process, e.g., a hybridization process for a hybridization solution, can be more than a few hours, during which there are bubbles formed at the end of the incubation process. The present disclosure provides a hybridization method with reduced bubble formation at the end of the hybridization process.

### CROSS-REFERENCE

This application claims the benefit of U.S. Provisional Patent Application No. 62/915,079, filed Oct. 15, 2019, which application is entirely incorporated herein by reference.

### BACKGROUND

Many biological experiments require long incubation time with different reagents and biological molecules kept in a container for a specific length of time. Because incubation experiments, e.g., a hybridization assay, rely on a complex and intertwined series of intermolecular reactions, e.g., different molecular associations in various hybridization steps, the experiments can take a long time to complete, e.g., an overnight hybridization experiment. For example, nucleic acid hybridizations are commonly used in biochemical research and diagnostic assays. In an example when conducting nucleic acid hybridization reactions, a labeled single-stranded target nucleic acid can be hybridized to a labeled or non-labeled nucleic acid probe, and the resulting nucleic acid duplexes can be detected by various methods. The incubation time for such incubations may require more than a few hours, e.g., more than 12 hours. Other incubation experiments may include carbohydrates, proteins, enzymes or peptides as example of the biological molecules to be tested/processed. Detection of the biological targets after the incubation step may be affected by changes to the assay environment during the incubation steps, e.g., the hybridization steps.

### SUMMARY

One aspect herein is a hybridization method with reduced bubble formation, the method comprising: centrifuging a hybridization solution disposed in a plurality of wells at a first speed for a first centrifuging time period; incubating the hybridization solution at a first temperature for a first incubation time period; centrifuging the hybridization solution at a second speed for second centrifuging time period; and incubating the hybridization solution at a second temperature for a second incubation time period; wherein at most 1% of the plurality of wells have a bubble within the hybridization solution.

In some embodiments, the first speed is about 1,875 rpm to about 7,500 rpm. In some embodiments, the first speed is about 1,875 rpm to about 2,000 rpm, about 1,875 rpm to about 2,500 rpm, about 1,875 rpm to about 3,000 rpm, about 1,875 rpm to about 3,500 rpm, about 1,875 rpm to about 4,000 rpm, about 1,875 rpm to about 4,500 rpm, about 1,875 rpm to about 5,000 rpm, about 1,875 rpm to about 5,500 rpm, about 1,875 rpm to about 6,000 rpm, about 1,875 rpm to about 6,500 rpm, about 1,875 rpm to about 7,500 rpm, about 2,000 rpm to about 2,500 rpm, about 2,000 rpm to about 3,000 rpm, about 2,000 rpm to about 3,500 rpm, about 2,000 rpm to about 4,000 rpm, about 2,000 rpm to about 4,500 rpm, about 2,000 rpm to about 5,000 rpm, about 2,000 rpm to about 5,500 rpm, about 2,000 rpm to about 6,000 rpm, about 2,000 rpm to about 6,500 rpm, about 2,000 rpm to about 7,500 rpm, about 2,500 rpm to about 3,000 rpm, about 2,500 rpm to about 3,500 rpm, about 2,500 rpm to about 4,000 rpm, about 2,500 rpm to about 4,500 rpm, about 2,500 rpm to about 5,000 rpm, about 2,500 rpm to about 5,500 rpm, about 2,500 rpm to about 6,000 rpm, about 2,500 rpm to about 6,500 rpm, about 2,500 rpm to about 7,500 rpm, about 3,000 rpm to about 3,500 rpm, about 3,000 rpm to about 4,000 rpm, about 3,000 rpm to about 4,500 rpm, about 3,000 rpm to about 5,000 rpm, about 3,000 rpm to about 5,500 rpm, about 3,000 rpm to about 6,000 rpm, about 3,000 rpm to about 6,500 rpm, about 3,000 rpm to about 7,500 rpm, about 3,500 rpm to about 4,000 rpm, about 3,500 rpm to about 4,500 rpm, about 3,500 rpm to about 5,000 rpm, about 3,500 rpm to about 5,500 rpm, about 3,500 rpm to about 6,000 rpm, about 3,500 rpm to about 6,500 rpm, about 3,500 rpm to about 7,500 rpm, about 4,000 rpm to about 4,500 rpm, about 4,000 rpm to about 5,000 rpm, about 4,000 rpm to about 5,500 rpm, about 4,000 rpm to about 6,000 rpm, about 4,000 rpm to about 6,500 rpm, about 4,000 rpm to about 7,500 rpm, about 4,500 rpm to about 5,000 rpm, about 4,500 rpm to about 5,500 rpm, about 4,500 rpm to about 6,000 rpm, about 4,500 rpm to about 6,500 rpm, about 4,500 rpm to about 7,500 rpm, about 5,000 rpm to about 5,500 rpm, about 5,000 rpm to about 6,000 rpm, about 5,000 rpm to about 6,500 rpm, about 5,000 rpm to about 7,500 rpm, about 5,500 rpm to about 6,000 rpm, about 5,500 rpm to about 6,500 rpm, about 5,500 rpm to about 7,500 rpm, about 6,000 rpm to about 6,500 rpm, about 6,000 rpm to about 7,500 rpm, or about 6,500 rpm to about 7,500 rpm. In some embodiments, the first speed is about 1,875 rpm, about 2,000 rpm, about 2,500 rpm, about 3,000 rpm, about 3,500 rpm, about 4,000 rpm, about 4,500 rpm, about 5,000 rpm, about 5,500 rpm, about 6,000 rpm, about 6,500 rpm, or about 7,500 rpm. In some embodiments, the first speed is at least about 1,875 rpm, about 2,000 rpm, about 2,500 rpm, about 3,000 rpm, about 3,500 rpm, about 4,000 rpm, about 4,500 rpm, about 5,000 rpm, about 5,500 rpm, about 6,000 rpm, or about 6,500 rpm. In some embodiments, the first speed is at most about 2,000 rpm, about 2,500 rpm, about 3,000 rpm, about 3,500 rpm, about 4,000 rpm, about 4,500 rpm, about 5,000 rpm, about 5,500 rpm, about 6,000 rpm, about 6,500 rpm, or about 7,500 rpm.

In some embodiments, the first centrifuging time period is about 0.5 minutes to about 2 minutes. In some embodiments, the first centrifuging time period is about 0.5 minutes to about 0.75 minutes, about 0.5 minutes to about 1 minute, about 0.5 minutes to about 1.25 minutes, about 0.5 minutes to about 1.5 minutes, about 0.5 minutes to about 1.75 minutes, about 0.5 minutes to about 2 minutes, about 0.75 minutes to about 1 minute, about 0.75 minutes to about 1.25 minutes, about 0.75 minutes to about 1.5 minutes, about 0.75 minutes to about 1.75 minutes, about 0.75 minutes to about 2 minutes, about 1 minute to about 1.25 minutes, about 1 minute to about 1.5 minutes, about 1 minute to about 1.75 minutes, about 1 minute to about 2 minutes, about 1.25 minutes to about 1.5 minutes, about 1.25 minutes to about 1.75 minutes, about 1.25 minutes to about 2 minutes, about 1.5 minutes to about 1.75 minutes, about 1.5 minutes to about 2 minutes, or about 1.75 minutes to about 2 minutes. In some embodiments, the first centrifuging time period is about 0.5 minutes, about 0.75 minutes, about 1 minute, about 1.25 minutes, about 1.5 minutes, about 1.75 minutes, or about 2 minutes. In some embodiments, the first centrifuging time period is at least about 0.5 minutes, about 0.75 minutes, about 1 minute, about 1.25 minutes, about 1.5 minutes, or about 1.75 minutes. In some embodiments, the first centrifuging time period is at most about 0.75 minutes, about 1 minute, about 1.25 minutes, about 1.5 minutes, about 1.75 minutes, or about 2 minutes.

In some embodiments, the first temperature is about 24° C. to about 92° C. In some embodiments, the first temperature is about 24° C. to about 30° C., about 24° C. to about 36° C., about 24° C. to about 42° C., about 24° C. to about 48° C., about 24° C. to about 54° C., about 24° C. to about 60° C., about 24° C. to about 66° C., about 24° C. to about 72° C., about 24° C. to about 78° C., about 24° C. to about 86° C., about 24° C. to about 92° C., about 30° C. to about 36° C., about 30° C. to about 42° C., about 30° C. to about 48° C., about 30° C. to about 54° C., about 30° C. to about 60° C., about 30° C. to about 66° C., about 30° C. to about 72° C., about 30° C. to about 78° C., about 30° C. to about 86° C., about 30° C. to about 92° C., about 36° C. to about 42° C., about 36° C. to about 48° C., about 36° C. to about 54° C., about 36° C. to about 60° C., about 36° C. to about 66° C., about 36° C. to about 72° C., about 36° C. to about 78° C., about 36° C. to about 86° C., about 36° C. to about 92° C., about 42° C. to about 48° C., about 42° C. to about 54° C., about 42° C. to about 60° C., about 42° C. to about 66° C., about 42° C. to about 72° C., about 42° C. to about 78° C., about 42° C. to about 86° C., about 42° C. to about 92° C., about 48° C. to about 54° C., about 48° C. to about 60° C., about 48° C. to about 66° C., about 48° C. to about 72° C., about 48° C. to about 78° C., about 48° C. to about 86° C., about 48° C. to about 92° C., about 54° C. to about 60° C., about 54° C. to about 66° C., about 54° C. to about 72° C., about 54° C. to about 78° C., about 54° C. to about 86° C., about 54° C. to about 92° C., about 60° C. to about 66° C., about 60° C. to about 72° C., about 60° C. to about 78° C., about 60° C. to about 86° C., about 60° C. to about 92° C., about 66° C. to about 72° C., about 66° C. to about 78° C., about 66° C. to about 86° C., about 66° C. to about 92° C., about 72° C. to about 78° C., about 72° C. to about 86° C., about 72° C. to about 92° C., about 78° C. to about 86° C., about 78° C. to about 92° C., or about 86° C. to about 92° C. In some embodiments, the first temperature is about 24° C., about 30° C., about 36° C., about 42° C., about 48° C., about 54° C., about 60° C., about 66° C., about 72° C., about 78° C., about 86° C., or about 92° C. In some embodiments, the first temperature is at least about 24° C., about 30° C., about 36° C., about 42° C., about 48° C., about 54° C., about 60° C., about 66° C., about 72° C., about 78° C., or about 86° C. In some embodiments, the first temperature is at most about 30° C., about 36° C., about 42° C., about 48° C., about 54° C., about 60° C., about 66° C., about 72° C., about 78° C., about 86° C., or about 92° C.

In some embodiments, the first incubation time is about 1 hour to about 6 hours. In some embodiments, the first incubation time is about 1 hour to about 1.5 hours, about 1 hour to about 2 hours, about 1 hour to about 2.5 hours, about 1 hour to about 3 hours, about 1 hour to about 3.5 hours, about 1 hour to about 4 hours, about 1 hour to about 4.5 hours, about 1 hour to about 5 hours, about 1 hour to about 5.5 hours, about 1 hour to about 6 hours, about 1.5 hours to about 2 hours, about 1.5 hours to about 2.5 hours, about 1.5 hours to about 3 hours, about 1.5 hours to about 3.5 hours, about 1.5 hours to about 4 hours, about 1.5 hours to about 4.5 hours, about 1.5 hours to about 5 hours, about 1.5 hours to about 5.5 hours, about 1.5 hours to about 6 hours, about 2 hours to about 2.5 hours, about 2 hours to about 3 hours, about 2 hours to about 3.5 hours, about 2 hours to about 4 hours, about 2 hours to about 4.5 hours, about 2 hours to about 5 hours, about 2 hours to about 5.5 hours, about 2 hours to about 6 hours, about 2.5 hours to about 3 hours, about 2.5 hours to about 3.5 hours, about 2.5 hours to about 4 hours, about 2.5 hours to about 4.5 hours, about 2.5 hours to about 5 hours, about 2.5 hours to about 5.5 hours, about 2.5 hours to about 6 hours, about 3 hours to about 3.5 hours, about 3 hours to about 4 hours, about 3 hours to about 4.5 hours, about 3 hours to about 5 hours, about 3 hours to about 5.5 hours, about 3 hours to about 6 hours, about 3.5 hours to about 4 hours, about 3.5 hours to about 4.5 hours, about 3.5 hours to about 5 hours, about 3.5 hours to about 5.5 hours, about 3.5 hours to about 6 hours, about 4 hours to about 4.5 hours, about 4 hours to about 5 hours, about 4 hours to about 5.5 hours, about 4 hours to about 6 hours, about 4.5 hours to about 5 hours, about 4.5 hours to about 5.5 hours, about 4.5 hours to about 6 hours, about 5 hours to about 5.5 hours, about 5 hours to about 6 hours, or about 5.5 hours to about 6 hours. In some embodiments, the first incubation time is about 1 hour, about 1.5 hours, about 2 hours, about 2.5 hours, about 3 hours, about 3.5 hours, about 4 hours, about 4.5 hours, about 5 hours, about 5.5 hours, or about 6 hours. In some embodiments, the first incubation time is at least about 1 hour, about 1.5 hours, about 2 hours, about 2.5 hours, about 3 hours, about 3.5 hours, about 4 hours, about 4.5 hours, about 5 hours, or about 5.5 hours. In some embodiments, the first incubation time is at most about 1.5 hours, about 2 hours, about 2.5 hours, about 3 hours, about 3.5 hours, about 4 hours, about 4.5 hours, about 5 hours, about 5.5 hours, or about 6 hours.

In some embodiments, the second speed is about 1,875 rpm to about 7,500 rpm. In some embodiments, the second speed is about 1,875 rpm to about 2,000 rpm, about 1,875 rpm to about 2,500 rpm, about 1,875 rpm to about 3,000 rpm, about 1,875 rpm to about 3,500 rpm, about 1,875 rpm to about 4,000 rpm, about 1,875 rpm to about 4,500 rpm, about 1,875 rpm to about 5,000 rpm, about 1,875 rpm to about 5,500 rpm, about 1,875 rpm to about 6,000 rpm, about 1,875 rpm to about 6,500 rpm, about 1,875 rpm to about 7,500 rpm, about 2,000 rpm to about 2,500 rpm, about 2,000 rpm to about 3,000 rpm, about 2,000 rpm to about 3,500 rpm, about 2,000 rpm to about 4,000 rpm, about 2,000 rpm to about 4,500 rpm, about 2,000 rpm to about 5,000 rpm, about 2,000 rpm to about 5,500 rpm, about 2,000 rpm to about 6,000 rpm, about 2,000 rpm to about 6,500 rpm, about 2,000 rpm to about 7,500 rpm, about 2,500 rpm to about 3,000 rpm, about 2,500 rpm to about 3,500 rpm, about 2,500 rpm to about 4,000 rpm, about 2,500 rpm to about 4,500 rpm, about 2,500 rpm to about 5,000 rpm, about 2,500 rpm to about 5,500 rpm, about 2,500 rpm to about 6,000 rpm, about 2,500 rpm to about 6,500 rpm, about 2,500 rpm to about 7,500 rpm, about 3,000 rpm to about 3,500 rpm, about 3,000 rpm to about 4,000 rpm, about 3,000 rpm to about 4,500 rpm, about 3,000 rpm to about 5,000 rpm, about 3,000 rpm to about 5,500 rpm, about 3,000 rpm to about 6,000 rpm, about 3,000 rpm to about 6,500 rpm, about 3,000 rpm to about 7,500 rpm, about 3,500 rpm to about 4,000 rpm, about 3,500 rpm to about 4,500 rpm, about 3,500 rpm to about 5,000 rpm, about 3,500 rpm to about 5,500 rpm, about 3,500 rpm to about 6,000 rpm, about 3,500 rpm to about 6,500 rpm, about 3,500 rpm to about 7,500 rpm, about 4,000 rpm to about 4,500 rpm, about 4,000 rpm to about 5,000 rpm, about 4,000 rpm to about 5,500 rpm, about 4,000 rpm to about 6,000 rpm, about 4,000 rpm to about 6,500 rpm, about 4,000 rpm to about 7,500 rpm, about 4,500 rpm to about 5,000 rpm, about 4,500 rpm to about 5,500 rpm, about 4,500 rpm to about 6,000 rpm, about 4,500 rpm to about 6,500 rpm, about 4,500 rpm to about 7,500 rpm, about 5,000 rpm to about 5,500 rpm, about 5,000 rpm to about 6,000 rpm, about 5,000 rpm to about 6,500 rpm, about 5,000 rpm to about 7,500 rpm, about 5,500 rpm to about 6,000 rpm, about 5,500 rpm to about 6,500 rpm, about 5,500 rpm to about 7,500 rpm, about 6,000 rpm to about 6,500 rpm, about 6,000 rpm to about 7,500 rpm, or about 6,500 rpm to about 7,500 rpm. In some embodiments, the second speed is about 1,875 rpm, about 2,000 rpm, about 2,500 rpm, about 3,000 rpm, about 3,500 rpm, about 4,000 rpm, about 4,500 rpm, about 5,000 rpm, about 5,500 rpm, about 6,000 rpm, about 6,500 rpm, or about 7,500 rpm. In some embodiments, the second speed is at least about 1,875 rpm, about 2,000 rpm, about 2,500 rpm, about 3,000 rpm, about 3,500 rpm, about 4,000 rpm, about 4,500 rpm, about 5,000 rpm, about 5,500 rpm, about 6,000 rpm, or about 6,500 rpm. In some embodiments, the second speed is at most about 2,000 rpm, about 2,500 rpm, about 3,000 rpm, about 3,500 rpm, about 4,000 rpm, about 4,500 rpm, about 5,000 rpm, about 5,500 rpm, about 6,000 rpm, about 6,500 rpm, or about 7,500 rpm.

In some embodiments, the second centrifuging time period is about 1 minute to about 10 minutes. In some embodiments, the second centrifuging time period is about 1 minute to about 2 minutes, about 1 minute to about 3 minutes, about 1 minute to about 4 minutes, about 1 minute to about 5 minutes, about 1 minute to about 6 minutes, about 1 minute to about 7 minutes, about 1 minute to about 8 minutes, about 1 minute to about 9 minutes, about 1 minute to about 10 minutes, about 2 minutes to about 3 minutes, about 2 minutes to about 4 minutes, about 2 minutes to about 5 minutes, about 2 minutes to about 6 minutes, about 2 minutes to about 7 minutes, about 2 minutes to about 8 minutes, about 2 minutes to about 9 minutes, about 2 minutes to about 10 minutes, about 3 minutes to about 4 minutes, about 3 minutes to about 5 minutes, about 3 minutes to about 6 minutes, about 3 minutes to about 7 minutes, about 3 minutes to about 8 minutes, about 3 minutes to about 9 minutes, about 3 minutes to about 10 minutes, about 4 minutes to about 5 minutes, about 4 minutes to about 6 minutes, about 4 minutes to about 7 minutes, about 4 minutes to about 8 minutes, about 4 minutes to about 9 minutes, about 4 minutes to about 10 minutes, about 5 minutes to about 6 minutes, about 5 minutes to about 7 minutes, about 5 minutes to about 8 minutes, about 5 minutes to about 9 minutes, about 5 minutes to about 10 minutes, about 6 minutes to about 7 minutes, about 6 minutes to about 8 minutes, about 6 minutes to about 9 minutes, about 6 minutes to about 10 minutes, about 7 minutes to about 8 minutes, about 7 minutes to about 9 minutes, about 7 minutes to about 10 minutes, about 8 minutes to about 9 minutes, about 8 minutes to about 10 minutes, or about 9 minutes to about 10 minutes. In some embodiments, the second centrifuging time period is about 1 minute, about 2 minutes, about 3 minutes, about 4 minutes, about 5 minutes, about 6 minutes, about 7 minutes, about 8 minutes, about 9 minutes, or about 10 minutes. In some embodiments, the second centrifuging time period is at least about 1 minute, about 2 minutes, about 3 minutes, about 4 minutes, about 5 minutes, about 6 minutes, about 7 minutes, about 8 minutes, or about 9 minutes. In some embodiments, the second centrifuging time period is at most about 2 minutes, about 3 minutes, about 4 minutes, about 5 minutes, about 6 minutes, about 7 minutes, about 8 minutes, about 9 minutes, or about 10 minutes.

In some embodiments, the second temperature is about 24° C. to about 92° C. In some embodiments, the second temperature is about 24° C. to about 30° C., about 24° C. to about 36° C., about 24° C. to about 42° C., about 24° C. to about 48° C., about 24° C. to about 54° C., about 24° C. to about 60° C., about 24° C. to about 66° C., about 24° C. to about 72° C., about 24° C. to about 78° C., about 24° C. to about 86° C., about 24° C. to about 92° C., about 30° C. to about 36° C., about 30° C. to about 42° C., about 30° C. to about 48° C., about 30° C. to about 54° C., about 30° C. to about 60° C., about 30° C. to about 66° C., about 30° C. to about 72° C., about 30° C. to about 78° C., about 30° C. to about 86° C., about 30° C. to about 92° C., about 36° C. to about 42° C., about 36° C. to about 48° C., about 36° C. to about 54° C., about 36° C. to about 60° C., about 36° C. to about 66° C., about 36° C. to about 72° C., about 36° C. to about 78° C., about 36° C. to about 86° C., about 36° C. to about 92° C., about 42° C. to about 48° C., about 42° C. to about 54° C., about 42° C. to about 60° C., about 42° C. to about 66° C., about 42° C. to about 72° C., about 42° C. to about 78° C., about 42° C. to about 86° C., about 42° C. to about 92° C., about 48° C. to about 54° C., about 48° C. to about 60° C., about 48° C. to about 66° C., about 48° C. to about 72° C., about 48° C. to about 78° C., about 48° C. to about 86° C., about 48° C. to about 92° C., about 54° C. to about 60° C., about 54° C. to about 66° C., about 54° C. to about 72° C., about 54° C. to about 78° C., about 54° C. to about 86° C., about 54° C. to about 92° C., about 60° C. to about 66° C., about 60° C. to about 72° C., about 60° C. to about 78° C., about 60° C. to about 86° C., about 60° C. to about 92° C., about 66° C. to about 72° C., about 66° C. to about 78° C., about 66° C. to about 86° C., about 66° C. to about 92° C., about 72° C. to about 78° C., about 72° C. to about 86° C., about 72° C. to about 92° C., about 78° C. to about 86° C., about 78° C. to about 92° C., or about 86° C. to about 92° C. In some embodiments, the second temperature is about 24° C., about 30° C., about 36° C., about 42° C., about 48° C., about 54° C., about 60° C., about 66° C., about 72° C., about 78° C., about 86° C., or about 92° C. In some embodiments, the second temperature is at least about 24° C., about 30° C., about 36° C., about 42° C., about 48° C., about 54° C., about 60° C., about 66° C., about 72° C., about 78° C., or about 86° C. In some embodiments, the second temperature is at most about 30° C., about 36° C., about 42° C., about 48° C., about 54° C., about 60° C., about 66° C., about 72° C., about 78° C., about 86° C., or about 92° C.

In some embodiments, the second incubation time is about 9 hours to about 36 hours. In some embodiments, the second incubation time is about 9 hours to about 12 hours, about 9 hours to about 15 hours, about 9 hours to about 18 hours, about 9 hours to about 21 hours, about 9 hours to about 24 hours, about 9 hours to about 27 hours, about 9 hours to about 30 hours, about 9 hours to about 33 hours, about 9 hours to about 36 hours, about 12 hours to about 15 hours, about 12 hours to about 18 hours, about 12 hours to about 21 hours, about 12 hours to about 24 hours, about 12 hours to about 27 hours, about 12 hours to about 30 hours, about 12 hours to about 33 hours, about 12 hours to about 36 hours, about 15 hours to about 18 hours, about 15 hours to about 21 hours, about 15 hours to about 24 hours, about 15 hours to about 27 hours, about 15 hours to about 30 hours, about 15 hours to about 33 hours, about 15 hours to about 36 hours, about 18 hours to about 21 hours, about 18 hours to about 24 hours, about 18 hours to about 27 hours, about 18 hours to about 30 hours, about 18 hours to about 33 hours, about 18 hours to about 36 hours, about 21 hours to about 24 hours, about 21 hours to about 27 hours, about 21 hours to about 30 hours, about 21 hours to about 33 hours, about 21 hours to about 36 hours, about 24 hours to about 27 hours, about 24 hours to about 30 hours, about 24 hours to about 33 hours, about 24 hours to about 36 hours, about 27 hours to about 30 hours, about 27 hours to about 33 hours, about 27 hours to about 36 hours, about 30 hours to about 33 hours, about 30 hours to about 36 hours, or about 33 hours to about 36 hours. In some embodiments, the second incubation time is about 9 hours, about 12 hours, about 15 hours, about 18 hours, about 21 hours, about 24 hours, about 27 hours, about 30 hours, about 33 hours, or about 36 hours. In some embodiments, the second incubation time is at least about 9 hours, about 12 hours, about 15 hours, about 18 hours, about 21 hours, about 24 hours, about 27 hours, about 30 hours, or about 33 hours. In some embodiments, the second incubation time is at most about 12 hours, about 15 hours, about 18 hours, about 21 hours, about 24 hours, about 27 hours, about 30 hours, about 33 hours, or about 36 hours.

In some embodiments, the first centrifuging time period is equal to the second centrifuging time period. In some embodiments, the first temperature is equal to the second temperature. In some embodiments, the first incubation time is equal to the second incubation time. In some embodiments, the hybridization solution comprises dimethyl sulfoxide (DMSO), polyoxyethylene (20) sorbitan monolaurate (Tween-20), 2-amino-2-(hydroxymethyl)propane-1,3-diol and buffer (Tris) and ethylenediaminetetraacetic acid (EDTA) buffer (Tris-EDTA buffer), tetramethylammonium chloride (TMAC), and water, or any combination thereof. In some embodiments, Tris-EDTA buffer has a pH of about 8. In some embodiments, the TMAC has a concentration of about 1 M, 2 M, 3 M, 4 M or 5 M in the hybridization solution. In some embodiments, the TMAC has a concentration of about 3 M in the hybridization solution.

In some embodiments, a volume percentage of the DMSO in the hybridization solution is about 5% to about 20%. In some embodiments, a volume percentage of the DMSO in the hybridization solution is about 5% to about 9%, about 5% to about 7%, about 5% to about 8%, about 5% to about 9%, about 5% to about 10%, about 5% to about 12%, about 5% to about 16%, about 5% to about 16%, about 5% to about 18%, about 5% to about 20%, about 9% to about 7%, about 9% to about 8%, about 9% to about 9%, about 9% to about 10%, about 9% to about 12%, about 9% to about 16%, about 9% to about 16%, about 9% to about 18%, about 9% to about 20%, about 7% to about 8%, about 7% to about 9%, about 7% to about 10%, about 7% to about 12%, about 7% to about 16%, about 7% to about 16%, about 7% to about 18%, about 7% to about 20%, about 8% to about 9%, about 8% to about 10%, about 8% to about 12%, about 8% to about 16%, about 8% to about 16%, about 8% to about 18%, about 8% to about 20%, about 9% to about 10%, about 9% to about 12%, about 9% to about 16%, about 9% to about 16%, about 9% to about 18%, about 9% to about 20%, about 10% to about 12%, about 10% to about 16%, about 10% to about 16%, about 10% to about 18%, about 10% to about 20%, about 12% to about 16%, about 12% to about 16%, about 12% to about 18%, about 12% to about 20%, about 16% to about 16%, about 16% to about 18%, about 16% to about 20%, about 16% to about 18%, about 16% to about 20%, or about 18% to about 20%. In some embodiments, a volume percentage of the DMSO in the hybridization solution is about 5%, about 9%, about 7%, about 8%, about 9%, about 10%, about 12%, about 16%, about 16%, about 18%, or about 20%. In some embodiments, a volume percentage of the DMSO in the hybridization solution is at least about 5%, about 9%, about 7%, about 8%, about 9%, about 10%, about 12%, about 16%, about 16%, or about 18%. In some embodiments, a volume percentage of the DMSO in the hybridization solution is at most about 9%, about 7%, about 8%, about 9%, about 10%, about 12%, about 16%, about 16%, about 18%, or about 20%.

In some embodiments, a volume percentage of the Tween-20 in the hybridization solution is about 0.025% to about 0.1%. In some embodiments, a volume percentage of the Tween-20 in the hybridization solution is about 0.025% to about 0.0375%, about 0.025% to about 0.05%, about 0.025% to about 0.0625%, about 0.025% to about 0.075%, about 0.025% to about 0.0875%, about 0.025% to about 0.1%, about 0.0375% to about 0.05%, about 0.0375% to about 0.0625%, about 0.0375% to about 0.075%, about 0.0375% to about 0.0875%, about 0.0375% to about 0.1%, about 0.05% to about 0.0625%, about 0.05% to about 0.075%, about 0.05% to about 0.0875%, about 0.05% to about 0.1%, about 0.0625% to about 0.075%, about 0.0625% to about 0.0875%, about 0.0625% to about 0.1%, about 0.075% to about 0.0875%, about 0.075% to about 0.1%, or about 0.0875% to about 0.1%. In some embodiments, a volume percentage of the Tween-20 in the hybridization solution is about 0.025%, about 0.0375%, about 0.05%, about 0.0625%, about 0.075%, about 0.0875%, or about 0.1%. In some embodiments, a volume percentage of the Tween-20 in the hybridization solution is at least about 0.025%, about 0.0375%, about 0.05%, about 0.0625%, about 0.075%, or about 0.0875%. In some embodiments, a volume percentage of the Tween-20 in the hybridization solution is at most about 0.0375%, about 0.05%, about 0.0625%, about 0.075%, about 0.0875%, or about 0.1%.

In some embodiments, a concentration of the Tris-EDTA in the hybridization solution is about 0.02 M to about 0.1 M for Tris, and from about 0.5 mM to about 2 mM for EDTA. In some embodiments, a concentration of the Tris in the hybridization solution is about 0.02 M to about 0.03 M, about 0.02 M to about 0.04 M, about 0.02 M to about 0.05 M, about 0.02 M to about 0.06 M, about 0.02 M to about 0.07 M, about 0.02 M to about 0.08 M, about 0.02 M to about 0.09 M, about 0.02 M to about 0.1 M, about 0.03 M to about 0.04 M, about 0.03 M to about 0.05 M, about 0.03 M to about 0.06 M, about 0.03 M to about 0.07 M, about 0.03 M to about 0.08 M, about 0.03 M to about 0.09 M, about 0.03 M to about 0.1 M, about 0.04 M to about 0.05 M, about 0.04 M to about 0.06 M, about 0.04 M to about 0.07 M, about 0.04 M to about 0.08 M, about 0.04 M to about 0.09 M, about 0.04 M to about 0.1 M, about 0.05 M to about 0.06 M, about 0.05 M to about 0.07 M, about 0.05 M to about 0.08 M, about 0.05 M to about 0.09 M, about 0.05 M to about 0.1 M, about 0.06 M to about 0.07 M, about 0.06 M to about 0.08 M, about 0.06 M to about 0.09 M, about 0.06 M to about 0.1 M, about 0.07 M to about 0.08 M, about 0.07 M to about 0.09 M, about 0.07 M to about 0.1 M, about 0.08 M to about 0.09 M, about 0.08 M to about 0.1 M, or about 0.09 M to about 0.1 M. In some embodiments, a concentration of Tris in the hybridization solution is about 0.02 M, about 0.03 M, about 0.04 M, about 0.05 M, about 0.06 M, about 0.07 M, about 0.08 M, about 0.09 M, or about 0.1 M. In some embodiments, a concentration of Tris in the hybridization solution is at least 0.02 M, about 0.03 M, about 0.04 M, about 0.05 M, about 0.06 M, about 0.07 M, or about 0.08 M, about 0.09 M. In some embodiments, a concentration of Tris in the hybridization solution is at most about 0.03 M, about 0.04 M, about 0.05 M, about 0.06 M, about 0.07 M, or about 0.08 M, about 0.09 M, or about 0.1 M.

In some embodiments, a concentration of the EDTA in the hybridization solution is about 0.5 mM to about 0.6 mM, about 0.5 mM to about 0.7 mM, about 0.5 mM to about 0.8 mM, about 0.5 mM to about 0.9 mM, about 0.5 mM to about 1.0 mM, about 0.5 mM to about 1.1 mM, about 0.5 mM to about 1.2 mM, about 0.5 mM to about 1.3 mM, about 0.5 mM to about 1.4 mM, about 0.5 mM to about 1.5 mM, about 0.5 mM to about 1.6 mM, about 0.5 mM to about 1.7 mM, about 0.5 mM to about 1.8 mM, about 0.5 mM to about 1.9 mM, about 0.5 mM to about 2.0 mM, about 0.6 mM to about 0.7 mM, about 0.6 mM to about 0.8 mM, about 0.6 mM to about 0.9 mM, about 0.6 mM to about 1.0 mM, about 0.6 mM to about 1.1 mM, about 0.6 mM to about 1.2 mM, about 0.6 mM to about 1.3 mM, about 0.6 mM to about 1.4 mM, about 0.6 mM to about 1.5 mM, about 0.6 mM to about 1.6 mM, about 0.6 mM to about 1.7 mM, about 0.6 mM to about 1.8 mM, about 0.6 mM to about 1.9 mM, about 0.6 mM to about 2.0 mM, about 0.7 mM to about 0.8 mM, about 0.7 mM to about 0.9 mM, about 0.7 mM to about 1.0 mM, about 0.7 mM to about 1.1 mM, about 0.7 mM to about 1.2 mM, about 0.7 mM to about 1.3 mM, about 0.7 mM to about 1.4 mM, about 0.7 mM to about 1.5 mM, about 0.7 mM to about 1.6 mM, about 0.7 mM to about 1.7 mM, about 0.7 mM to about 1.8 mM, about 0.7 mM to about 1.9 mM, about 0.7 mM to about 2.0 mM, about 0.8 mM to about 0.9 mM, about 0.8 mM to about 1.0 mM, about 0.8 mM to about 1.1 mM, about 0.8 mM to about 1.2 mM, about 0.8 mM to about 1.3 mM, about 0.8 mM to about 1.4 mM, about 0.8 mM to about 1.5 mM, about 0.8 mM to about 1.6 mM, about 0.8 mM to about 1.7 mM, about 0.8 mM to about 1.8 mM, about 0.8 mM to about 1.9 mM, about 0.8 mM to about 2.0 mM, about 0.9 mM to about 1.0 mM, about 0.9 mM to about 1.1 mM, about 0.9 mM to about 1.2 mM, about 0.9 mM to about 1.3 mM, about 0.9 mM to about 1.4 mM, about 0.9 mM to about 1.5 mM, about 0.9 mM to about 1.6 mM, about 0.9 mM to about 1.7 mM, about 0.9 mM to about 1.8 mM, about 0.9 mM to about 1.9 mM, about 0.9 mM to about 2.0 mM, about 1.0 mM to about 1.1 mM, about 1.0 mM to about 1.2 mM, about 1.0 mM to about 1.3 mM, about 1.0 mM to about 1.4 mM, about 1.0 mM to about 1.5 mM, about 1.0 mM to about 1.6 mM, about 1.0 mM to about 1.7 mM, about 1.0 mM to about 1.8 mM, about 1.0 mM to about 1.9 mM, about 1.0 mM to about 2.0 mM, about 1.1 mM to about 1.2 mM, about 1.1 mM to about 1.3 mM, about 1.1 mM to about 1.4 mM, about 1.1 mM to about 1.5 mM, about 1.1 mM to about 1.6 mM, about 1.1 mM to about 1.7 mM, about 1.1 mM to about 1.8 mM, about 1.1 mM to about 1.9 mM, about 1.1 mM to about 2.0 mM, about 1.2 mM to about 1.3 mM, about 1.2 mM to about 1.4 mM, about 1.2 mM to about 1.5 mM, about 1.2 mM to about 1.6 mM, about 1.2 mM to about 1.7 mM, about 1.2 mM to about 1.8 mM, about 1.2 mM to about 1.9 mM, about 1.2 mM to about 2.0 mM, 1.2 mM to about 1.4 mM, about 1.3 mM to about 1.5 mM, about 1.3 mM to about 1.6 mM, about 1.3 mM to about 1.7 mM, about 1.3 mM to about 1.8 mM, about 1.3 mM to about 1.9 mM, about 1.3 mM to about 2.0 mM, about 1.4 mM to about 1.5 mM, about 1.4 mM to about 1.6 mM, about 1.4 mM to about 1.7 mM, about 1.4 mM to about 1.8 mM, about 1.4 mM to about 1.9 mM, about 1.4 mM to about 2.0 mM, about 1.5 mM to about 1.6 mM, about 1.5 mM to about 1.7 mM, about 1.5 mM to about 1.8 mM, about 1.5 mM to about 1.9 mM, about 1.5 mM to about 2.0 mM, about 1.6 mM to about 1.7 mM, about 1.6 mM to about 1.8 mM, about 1.6 mM to about 1.9 mM, about 1.6 mM to about 2.0 mM, about 1.76 mM to about 1.8 mM, about 1.7 mM to about 1.9 mM, about 1.7 mM to about 2.0 mM, about 1.8 mM to about 1.9 mM, about 1.8 mM to about 2.0 mM, or about 1.9 mM to about 2.0 mM. In some embodiments, a concentration of EDTA in the hybridization solution is about 0.5 mM, about 0.6 mM, about 0.7 mM, about 0.8 mM, about 0.9 mM, about 1.0 mM, about 1.1 mM, about 1.2 mM, about 1.3 mM, about 1.4 mM, about 1.5 mM, about 1.6 mM, about 1.7 mM, about 1.8 mM, about 1.9 mM, or about 2.0 mM. In some embodiments, a concentration of EDTA in the hybridization solution is at least about 0.5 mM, about 0.6 mM, about 0.7 mM, about 0.8 mM, about 0.9 mM, about 1.0 mM, about 1.1 mM, about 1.2 mM, about 1.3 mM, about 1.4 mM, about 1.5 mM, about 1.6 mM, about 1.7 mM, about 1.8 mM, or about 1.9 mM. In some embodiments, a concentration of EDTA in the hybridization solution is at most about 0.6 mM, about 0.7 mM, about 0.8 mM, about 0.9 mM, about 1.0 mM, about 1.1 mM, about 1.2 mM, about 1.3 mM, about 1.4 mM, about 1.5 mM, about 1.6 mM, about 1.7 mM, about 1.8 mM, about 1.9 mM, or about 2.0 mM.

In some embodiments, a molar ratio of Tris:EDTA in the hybridization solution is about 20 to about 100. In some embodiments, a molar ratio of Tris:EDTA in the hybridization solution is about 20 to about 30, about 20 to about 40, about 20 to about 50, about 20 to about 60, about 20 to about 70, about 20 to about 80, about 20 to about 90, about 20 to about 100, about 30 to about 40, about 30 to about 50, about 30 to about 60, about 30 to about 70, about 30 to about 80, about 30 to about 90, about 30 to about 100, about 40 to about 50, about 40 to about 60, about 40 to about 70, about 40 to about 80, about 40 to about 90, about 40 to about 100, about 50 to about 60, about 50 to about 70, about 50 to about 80, about 50 to about 90, about 50 to about 100, about 60 to about 70, about 60 to about 80, about 60 to about 90, about 60 to about 100, about 70 to about 80, about 70 to about 90, about 70 to about 100, about 80 to about 90, about 80 to about 100, or about 90 to about 100. In some embodiments, a molar ratio of Tris:EDTA in the hybridization solution is about 20, about 30, about 40, about 50, about 60, about 70, about 80, about 90, or about 100. In some embodiments, a molar ratio of Tris:EDTA in the hybridization solution is at least about 20, about 30, about 40, about 50, about 60, about 70, about 80, or about 90. In some embodiments, a molar ratio of Tris:EDTA in the hybridization solution is at most about 30, about 40, about 50, about 60, about 70, about 80, about 90, or about 100.

In some embodiments, a volume percentage of the TMAC in the hybridization solution is about 30% to about 99%. In some embodiments, a volume percentage of the TMAC in the hybridization solution is about 30% to about 35%, about 30% to about 40%, about 30% to about 45%, about 30% to about 50%, about 30% to about 55%, about 30% to about 60%, about 30% to about 65%, about 30% to about 70%, about 30% to about 80%, about 30% to about 90%, about 30% to about 99%, about 35% to about 40%, about 35% to about 45%, about 35% to about 50%, about 35% to about 55%, about 35% to about 60%, about 35% to about 65%, about 35% to about 70%, about 35% to about 80%, about 35% to about 90%, about 35% to about 99%, about 40% to about 45%, about 40% to about 50%, about 40% to about 55%, about 40% to about 60%, about 40% to about 65%, about 40% to about 70%, about 40% to about 80%, about 40% to about 90%, about 40% to about 99%, about 45% to about 50%, about 45% to about 55%, about 45% to about 60%, about 45% to about 65%, about 45% to about 70%, about 45% to about 80%, about 45% to about 90%, about 45% to about 99%, about 50% to about 55%, about 50% to about 60%, about 50% to about 65%, about 50% to about 70%, about 50% to about 80%, about 50% to about 90%, about 50% to about 99%, about 55% to about 60%, about 55% to about 65%, about 55% to about 70%, about 55% to about 80%, about 55% to about 90%, about 55% to about 99%, about 60% to about 65%, about 60% to about 70%, about 60% to about 80%, about 60% to about 90%, about 60% to about 99%, about 65% to about 70%, about 65% to about 80%, about 65% to about 90%, about 65% to about 99%, about 70% to about 80%, about 70% to about 90%, about 70% to about 99%, about 80% to about 90%, about 80% to about 99%, or about 90% to about 99%. In some embodiments, a volume percentage of the TMAC in the hybridization solution is about 30%, about 35%, about 40%, about 45%, about 50%, about 55%, about 60%, about 65%, about 70%, about 80%, about 90%, or about 99%. In some embodiments, a volume percentage of the TMAC in the hybridization solution is at least about 30%, about 35%, about 40%, about 45%, about 50%, about 55%, about 60%, about 65%, about 70%, about 80%, or about 90%. In some embodiments, a volume percentage of the TMAC in the hybridization solution is at most about 35%, about 40%, about 45%, about 50%, about 55%, about 60%, about 65%, about 70%, about 80%, about 90%, or about 99%.

In some embodiments, a concentration of the TMAC in the hybridization solution is about 2 M to about 4 M. In some embodiments, a concentration of the TMAC in the hybridization solution is about 2 M to about 2.2 M, about 2 M to about 2.4 M, about 2 M to about 2.6 M, about 2 M to about 2.8 M, about 2 M to about 3.0 M, is about 2 M to about 3.2 M, about 2 M to about 3.4 M, about 2 M to about 3.6 M, about 2 M to about 3.8 M, about 2 M to about 4.0 M, about 2.2 M to about 2.4 M, about 2.2 M to about 2.6 M, about 2.2 M to about 2.8 M, about 2.2 M to about 3.0 M, is about 2.2 M to about 3.2 M, about 2.2 M to about 3.4 M, about 2.2 M to about 3.6 M, about 2.2 M to about 3.8 M, about 2.2 M to about 4.0 M, about 2.4 M to about 2.6 M, about 2.4 M to about 2.8 M, about 2.4 M to about 3.0 M, is about 2.4 M to about 3.2 M, about 2.4 M to about 3.4 M, about 2.4 M to about 3.6 M, about 2.4 M to about 3.8 M, about 2.4 M to about 4.0 M, about 2.6 M to about 2.8 M, about 2.6 M to about 3.0 M, is about 2.6 M to about 3.2 M, about 2.6 M to about 3.4 M, about 2.6 M to about 3.6 M, about 2.6 M to about 3.8 M, about 2.6 M to about 4.0 M, about 2.8 M to about 3.0 M, is about 2.8 M to about 3.2 M, about 2.8 M to about 3.4 M, about 2.8 M to about 3.6 M, about 2.8 M to about 3.8 M, about 2.8 M to about 4.0 M, is about 3.0 M to about 3.2 M, about 3.0 M to about 3.4 M, about 3.0 M to about 3.6 M, about 3.0 M to about 3.8 M, about 3.0 M to about 4.0 M, about 3.2 M to about 3.4 M, about 3.2 M to about 3.6 M, about 3.2 M to about 3.8 M, about 3.2 M to about 4.0 M, about 3.4 M to about 3.6 M, about 3.4 M to about 3.8 M, about 3.4 to about 4.0 M, about 3.6 M to about 3.8 M, about 3.6 to about 4.0 M, or about 3.8 M to about 4.0 M. In some embodiments, a concentration of the TMAC in the hybridization solution is about 2 M, about 2.2 M, about 2.4 M, about 2.6 M, about 2.8 M, about 3.0 M, about 3.2 M, about 3.4 M, about 3.6 M, about 3.8 M, or about 4.0 M. In some embodiments, a concentration of the TMAC in the hybridization solution is at least about 2 M, about 2.2 M, about 2.4 M, about 2.6 M, about 2.8 M, about 3.0 M, about 3.2 M, about 3.4 M, about 3.6 M, or about 3.8 M. In some embodiments, a concentration of the TMAC in the hybridization solution is at most about 2.2 M, about 2.4 M, about 2.6 M, about 2.8 M, about 3.0 M, about 3.2 M, about 3.4 M, about 3.6 M, about 3.8 M, or about 4.0 M.

In some embodiments, a volume percentage of the water in the hybridization solution is about 12% to about 49%. In some embodiments, a concentration of the water in the hybridization solution is about 12% to about 16%, about 12% to about 20%, about 12% to about 24%, about 12% to about 28%, about 12% to about 32%, about 12% to about 36%, about 12% to about 40%, about 12% to about 44%, about 12% to about 48%, about 12% to about 49%, about 16% to about 20%, about 16% to about 24%, about 16% to about 28%, about 16% to about 32%, about 16% to about 36%, about 16% to about 40%, about 16% to about 44%, about 16% to about 48%, about 16% to about 49%, about 20% to about 24%, about 20% to about 28%, about 20% to about 32%, about 20% to about 36%, about 20% to about 40%, about 20% to about 44%, about 20% to about 48%, about 20% to about 49%, about 24% to about 28%, about 24% to about 32%, about 24% to about 36%, about 24% to about 40%, about 24% to about 44%, about 24% to about 48%, about 24% to about 49%, about 28% to about 32%, about 28% to about 36%, about 28% to about 40%, about 28% to about 44%, about 28% to about 48%, about 28% to about 49%, about 32% to about 36%, about 32% to about 40%, about 32% to about 44%, about 32% to about 48%, about 32% to about 49%, about 36% to about 40%, about 36% to about 44%, about 36% to about 48%, about 36% to about 49%, about 40% to about 44%, about 40% to about 48%, about 40% to about 49%, about 44% to about 48%, about 44% to about 49%, or about 48% to about 49%. In some embodiments, a concentration of the water in the hybridization solution is about 12%, about 16%, about 20%, about 24%, about 28%, about 32%, about 36%, about 40%, about 44%, about 48%, or about 49%. In some embodiments, a concentration of the water in the hybridization solution is at least about 12%, about 16%, about 20%, about 24%, about 28%, about 32%, about 36%, about 40%, about 44%, or about 48%. In some embodiments, a concentration of the water in the hybridization solution is at most about 16%, about 20%, about 24%, about 28%, about 32%, about 36%, about 40%, about 44%, about 48%, or about 49%.

In some embodiments, the water comprises nuclease-free water. In some embodiments, the bubble comprises an air bubble.

In some embodiments, a volume of the hybridization solution disposed within each of the plurality of wells is about 15 μL to about 60 μL. In some embodiments, a volume of the hybridization solution disposed within each of the plurality of wells is about 15 μL to about 20 μL, about 15 μL to about 25 μL, about 15 μL to about 30 μL, about 15 μL to about 35 μL, about 15 μL to about 40 μL, about 15 μL to about 45 μL, about 15 μL to about 50 μL, about 15 μL to about 55 μL, about 15 μL to about 60 μL, about 20 μL to about 25 μL, about 20 μL to about 30 μL, about 20 μL to about 35 μL, about 20 μL to about 40 μL, about 20 μL to about 45 μL, about 20 μL to about 50 μL, about 20 μL to about 55 μL, about 20 μL to about 60 μL, about 25 μL to about 30 μL, about 25 μL to about 35 μL, about 25 μL to about 40 μL, about 25 μL to about 45 μL, about 25 μL to about 50 μL, about 25 μL to about 55 μL, about 25 μL to about 60 μL, about 30 μL to about 35 μL, about 30 μL to about 40 μL, about 30 μL to about 45 μL, about 30 μL to about 50 μL, about 30 μL to about 55 μL, about 30 μL to about 60 μL, about 35 μL to about 40 μL, about 35 μL to about 45 μL, about 35 μL to about 50 μL, about 35 μL to about 55 μL, about 35 μL to about 60 μL, about 40 μL to about 45 μL, about 40 μL to about 50 μL, about 40 μL to about 55 μL, about 40 μL to about 60 μL, about 45 μL to about 50 μL, about 45 μL to about 55 μL, about 45 μL to about 60 μL, about 50 μL to about 55 μL, about 50 μL to about 60 μL, or about 55 μL to about 60 μL. In some embodiments, a volume of the hybridization solution disposed within each of the plurality of wells is about 15 μL, about 20 μL, about 25 μL, about 30 μL, about 35 μL, about 40 μL, about 45 μL, about 50 μL, about 55 μL, or about 60 μL. In some embodiments, a volume of the hybridization solution disposed within each of the plurality of wells is at least about 15 μL, about 20 μL, about 25 μL, about 30 μL, about 35 μL, about 40 μL, about 45 μL, about 50 μL, or about 55 μL. In some embodiments, a volume of the hybridization solution disposed within each of the plurality of wells is at most about 20 μL, about 25 μL, about 30 μL, about 35 μL, about 40 μL, about 45 μL, about 50 μL, about 55 μL, or about 60 μL.

Another aspect disclosed herein is a hybridization device comprising: a top clamping bracket, a bottom clamping bracket, one or more latches configured to clamp the top clamping bracket and the bottom clamping bracket together, a reaction chamber defined by the clamped top clamping bracket and the bottom clamping bracket, and a peg plate disposed within the reaction chamber, the peg plate comprises a plurality of pegs.

In some embodiments, the peg plate further comprises a plurality chips, each of the plurality of chips is on the top surface of a selected peg of the plurality of pegs. In some embodiments, the hybridization device further comprises a well plate disposed within the reaction chamber and mated with the peg plate, the well plate comprises a plurality of wells, each peg of the peg plate mates with a well of the well plate. In some embodiments, the top clamping bracket further comprises an open port or window. In some embodiments, the top clamping bracket further comprises a plurality of indentation on the top surface of the top clamping bracket. In some embodiments, the bottom clamping bracket further comprises a plurality of feet. In some embodiments, the number of indentations is the same as the number of feet. In some embodiments, there are 4 indentation and 4 feet. In some embodiments, the position and size of the indentations and feet are configured to match each other, thereby the hybridization device is configured to stack with another hybridization device having the same configuration of the hybridization device. In some embodiments, the hybridization device has two, three, or four latches.

Still another aspect disclosed here is a hybridization device comprising:
a) a top clamping bracket;
b) a bottom clamping bracket;
c) one or more latches configured to clamp the top clamping bracket and the bottom clamping bracket together;
d) a reaction chamber defined by the clamped top clamping bracket and the bottom clamping bracket;
e) a well plate disposed within the reaction chamber, the well plate comprising a plurality of wells; and
f) a peg plate disposed within the reaction chamber, the peg plate comprising a plurality of pegs;
wherein the peg plate mates with the well plate, thereby each peg of the plurality of pegs mates with a selected well of the plurality of wells.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows a flowchart of an example hybridization method, per some embodiments herein;

FIG. 2 shows an illustration of an example centrifuging machine, per some embodiments herein;

FIG. 3 shows an illustration of an example incubation device, per some embodiments herein; and

FIG. 4 shows an illustration of an example hybridization well, per some embodiments herein.

FIG. 5 shows an illustration of a perspective view of an example incubation device, per some embodiments herein;

FIG. 6 shows an illustration of a top view of an example incubation device, per some embodiments herein;

FIG. 7 shows an illustration of a side view of an example incubation device, per some embodiments herein; and

FIG. 8 shows an illustration of a widthwise cross-section view of an example incubation device, per some embodiments herein.

FIG. 9 shows an illustration of another widthwise cross-section view of an example incubation device, per some embodiments herein;

FIG. 10 shows an illustration of a lengthwise cross-section view of an example incubation device, per some embodiments herein;

FIG. 11 shows an illustration of another lengthwise cross-section view of an example incubation device, per some embodiments herein; and

FIG. 12 shows an illustration of a perspective view of an example peg plate model, per some embodiments herein.

FIG. 13 shows an illustration of a perspective view of an example 384 peg plate model with chips on it, per some embodiments herein.

### DETAILED DESCRIPTION

Loading and unloading of wells having a hybridization solution during hybridization and incubation of the hybridization solution often produces bubbles in the hybridization solution. Such bubbles are detrimental as they occlude the surface of the chips and thus prevent the hybridization reaction from taking place in those locations. While mechanical ‘tapping’ of the wells may dislodge some of these entrapped bubbles, such methods are not viable for large scale production.

### Hybridization Methods

One aspect herein is a hybridization method with reduced bubble formation. As shown in FIG. 1, the method comprises a first centrifuging of the hybridization solution 101, a first incubation of the hybridization solution 102, a second centrifuging of the hybridization solution 103, and a second incubation of the hybridization solution 104.

In some embodiments, the hybridization solution is disposed in a plurality of wells. In some embodiments, the hybridization solution is disposed in a plurality of wells during the first centrifuging of the hybridization solution 101, the first incubation of the hybridization solution 102, the second centrifuging of the hybridization solution 103, and the second incubation of the hybridization solution 104. In some embodiments, a volume of the hybridization solution disposed within each of the plurality of wells is about 15 μL to about 60 μL.

In some embodiments, at most 1% of the plurality of wells have a bubble within the hybridization solution after the method is performed. In other embodiments, at most 0.25%, 0.5%, 0.75%, 1%, 1.25%, 1.5%, 1.75%, or 2% of the plurality of wells have a bubble within the hybridization solution after the method is performed. In some embodiments, the bubble within the hybridization solution is visible to the naked eye. In some embodiments, the bubble within the hybridization solution is visible under a microscope.

In some embodiments, the first centrifuging of the hybridization solution 101 comprises centrifuging the hybridization solution at a first speed for a first centrifuging time period. In some embodiments, the first speed is about 1,875 rpm to about 7,500 rpm. In some embodiments, the first centrifuging time period is about 0.5 minutes to about 2 minutes. In some embodiments, at least one of the first centrifuging time and the first centrifuging speed reduce the formation of bubbles within the hybridization agent, such that less than 1% of the wells of the hybridization solution exhibit a bubble.

In some embodiments, the first incubation of the hybridization solution 102 comprises incubating the hybridization solution at a first temperature for a first incubation time period. In some embodiments, the first temperature is about 24° C. to about 92° C. In some embodiments, the first incubation time is about 1 hour to about 6 hours. In some embodiments, at least one of the first incubation time and the first incubation temperature reduce the formation of bubbles within the hybridization agent, such that less than 1% of the wells of the hybridization solution exhibit a bubble.

In some embodiments, the second centrifuging of the hybridization solution 103 comprises centrifuging the hybridization solution at a second speed for a second centrifuging time period. In some embodiments, the second speed is about 1,875 rpm to about 7,500 rpm. In some embodiments, the second centrifuging time period is about 1 minute to about 10 minutes. In some embodiments, at least one of the second centrifuging time and the second centrifuging speed reduce the formation of bubbles within the hybridization agent, such that less than 1% of the wells of the hybridization solution exhibit a bubble.

In some embodiments, the second incubation of the hybridization solution 104 comprises incubating the hybridization solution at a second temperature for a second incubation time period. In some embodiments, the second temperature is about 24° C. to about 92° C. In some embodiments, the second incubation time is about 9 hours to about 36 hours. In some embodiments, at least one of the second incubation time and the second incubation temperature reduce the formation of bubbles within the hybridization agent, such that less than 1% of the wells of the hybridization solution exhibit a bubble.

In some embodiments, the first centrifuging time period is equal to the second centrifuging time period. In some embodiments, the first temperature is equal to the second temperature. In some embodiments, the first incubation time is equal to the second incubation time. In some embodiments, at least one of the first centrifuging and the second centrifuging removes any bubbles from the hybridization solution. In some embodiments, at least one of the first centrifuging and the second centrifuging hinders nucleation of bubbles within the hybridization solution. In some embodiments, at least one of the first centrifuging and the second centrifuging hinders nucleation of bubbles within the hybridization solution during incubation.

In some embodiments, the hybridization solution comprises DMSO, a surfactant, Tris-EDTA, Tris-HCL, TMAC, water, or any combination thereof. In some embodiments, the surfactant comprises Tween-20. In some embodiments, the water comprises nuclease-free water. In some embodiments, at least one of the Tris-EDTA and the Tris-HCL have a pH of about 8. In some embodiments, the TMAC has a concentration of about 5M. In some embodiments, a concentration of the DMSO in the hybridization solution is about 5% to about 20%. In some embodiments, a concentration of the surfactant in the hybridization solution is about 0.25% to about 1%. In some embodiments, a concentration of the Tris-EDTA in the hybridization solution is about 0.5% to about 2%. In some embodiments, a concentration of the Tris-HCL in the hybridization solution is about 2% to about 8%. In some embodiments, a concentration of the TMAC in the hybridization solution is about 30% to about 99%. In some embodiments, a concentration of the water in the hybridization solution is about 12% to about 49%. The concentration of the DMSO, surfactant, Tris-EDTA, Tris-HCL, TMAC, and water within the hybridization solution represents percentage by mass or a percentage by volume. The concentration of the components within the hybridization solution can be measured by any standard methods. In some embodiments, the surfactant reduces the formation of bubbles within the hybridization agent, such that less than 1% of the wells of the hybridization solution exhibit a bubble.

In some embodiments, the surfactant reduces the formation of bubbles within the hybridization agent, such that less than 1% of the wells of the hybridization solution exhibit a bubble. In some embodiments, the surfactant enables the formation of a deep meniscus in the hybridization solution to entrap bubbles. In some embodiments, the concentration of the DMSO, surfactant, Tris-EDTA, Tris-HCL, TMAC, water, or any combination thereof in the hybridization solution reduces the formation of bubbles within the hybridization agent, such that less than 1% of the wells of the hybridization solution exhibit a bubble. In some embodiments, the bubble comprises an air bubble. In some embodiments, the bubble comprises a gaseous bubble.

### Hybridization Apparatus

One aspect herein is a hybridization method with reduced bubble formation comprising centrifuging and incubation of a hybridization solution. In some embodiments, the centrifuging of the hybridization solution can be performed with any mechanism capable of centrifuging the hybridization solution at a speed of at least about 1,875 rpm. One example centrifuging machine is shown in FIG. 2. In some embodiments, the incubation of the hybridization solution can be performed with any mechanism capable of centrifuging the hybridization solution at a temperature of at least about 24° C.

FIG. 3 shows an example illustration of a hybridization apparatus 300, which comprises a plurality of individual wells for the hybridization reactions, inside a chamber. FIG. 3 shows an image of an example well plate and chip plate that are clamped together and placed inside a chamber. In some embodiments, the well plate and the chip plate are clamped together during one or more of the first centrifuging of the hybridization solution, the first incubation of the hybridization solution, the second centrifuging of the hybridization solution, and the second incubation of the hybridization solution. In some embodiments, the wells are secured within the well plate by a clamp. In some embodiments, the clamp comprises, a screw, a bolt, a nut, a washer, a binder clip, or any combination thereof. In some embodiments when the well plate and the chip plate are placed in a chamber, the well plate and the chip plate are not clamped together by a clamp or binder clips.

FIG. 4 shows an example illustration of an individual hybridization well 400. As shown in FIG. 4 each individual hybridization well 400 comprises a well plate 401 comprising a well 401A, a hybridization solution 402 disposed within the well 401A, a chip plate 402, and a chip 403. In some embodiments, the hybridization solution 402 is disposed in a plurality of wells 401A during centrifuging of the hybridization solution 402, incubation of the hybridization solution 402, or both. In some embodiments, a volume of the hybridization solution 402 disposed within each of the plurality of wells 401A is about 15 μL to about 60 μL. In some embodiments, the well plate 401 and the chip plate 402 are clamped together during centrifuging of the hybridization solution 402, incubation of the hybridization solution 402, or both. In some embodiments, the well plate 401 and the chip plate 402 are not clamped together during the centrifuging of the hybridization solution 402 or the incubation of the hybridization solution 402, or neither. Without using the hybridization methods disclosed herein (i.e., not including a centrifuge step during or between the incubation steps), bubbles in the hybridization solution 402 occlude and prevent the hybridization reaction from taking place on a surface of the chip 403. When using the hybridization methods disclosed herein (i.e., including a centrifuge step during or between the incubation steps), the amount of the bubbles in the hybridization solution 402 formed on a surface of the chip 403 during the hybridization reactions is reduced when compared with the conditions when no centrifuge step is used during or between the incubation steps.

FIG. 5 shows an illustration of a perspective view of an example incubation device 500, per some embodiments herein. FIG. 5 shows the incubation device 500 may comprise a top clamping bracket 510, a bottom clamping bracket 520, more than one latch 530 that clamps the top clamping bracket 510 and the bottom clamping bracket 520 together, more than one rubber foot 540 on the bottom face of the bottom clamping bracket 520, and more than one indentation 550 on the top face of the top clamping bracket 510 for stacking. In addition, the incubation device 500 may comprise an open port or window 560 such that a technician may view the hybridization plate during the experiment, a top surface 570 of the bottom clamping bracket onto which the 384 peg plate can be clamped. Once clamped together by the top and bottom clamping brackets, the 384 peg plate can be securely fixed inside the incubation chamber residing between the two clamped brackets. The indentations on the top face of the top clamping bracket 510 may allow stacking another clamped plate during the incubation.

FIG. 6 shows an illustration of a top view of an example incubation device 600, per some embodiments herein. FIG. 6 shows the incubation device 600 may comprise a top clamping bracket 610, a bottom clamping bracket 620, more than one latch 630 that clamps the top clamping bracket 610 and the bottom clamping bracket 620 together, and more than one indentation 650 on the top face of the top clamping bracket 610 for stacking. In addition, the incubation device 600 may comprise an open port or window 660 such that a technician may view the hybridization plate during the experiment, a top surface 670 of the bottom clamping bracket onto which the 384 peg plate can be clamped. Once clamped together by the top and bottom clamping brackets, the 384 peg plate can be securely fixed inside the incubation chamber residing between the two clamped brackets. The indentations on the top face of the top clamping bracket 610 may allow stacking another clamped plate during the incubation.

FIG. 7 shows an illustration of a side view of an example incubation device 700, per some embodiments herein. FIG. 7 shows the incubation device 700 may comprise a top clamping bracket 710, a bottom clamping bracket 720, more than one latch 730 that clamps the top clamping bracket 710 and the bottom clamping bracket 720 together, and more than one rubber foot 740 on the bottom face of the bottom clamping bracket 720. Once clamped together by the top and bottom clamping brackets, the 384 peg plate can be securely fixed inside the incubation chamber residing between the two clamped brackets.

FIG. 8 shows an illustration of a widthwise cross-section view of an example incubation device 800, per some embodiments herein. FIG. 8 shows the incubation device 800 may comprise a top clamping bracket 810, a bottom clamping bracket 820, more than one latch 830 that clamps the top clamping bracket 810 and the bottom clamping bracket 820 together, and more than one rubber foot 840 on the bottom face of the bottom clamping bracket 820. Inside the incubation chamber is a pair of mated plates: 384 well pate 865 and 384 peg plate 875. Once clamped together by the top and bottom clamping brackets, the 384 well pate 865 and the 384 peg plate 875 can be securely fixed inside the incubation chamber residing between the two clamped brackets. The 384 peg plate 875 may comprise chips on top of each of its pegs such that the chip can reach into a well and interact with reagents in the well. A rubber gasket 855 can be placed around the peripheral of the mated plates and between the interface of the two mated plates, thereby providing a better seal.

FIG. 9 shows an illustration of another widthwise cross-section partial view of an example incubation device 900, per some embodiments herein. Some of the parts are removed for clarity. FIG. 9 shows the incubation device 900 may comprise a top clamping bracket 910, a bottom clamping bracket 920, more than one latch 930 that clamps the top clamping bracket 910 and the bottom clamping bracket 920 together, and more than one rubber foot 940 on the bottom face of the bottom clamping bracket 920. Inside the incubation chamber is a pair of mated plates: 384 well pate 965 and 384 peg plate 975. Once clamped together by the top and bottom clamping brackets, the 384 well pate 965 and the 384 peg plate 975 can be securely fixed inside the incubation chamber residing between the two clamped brackets. The 384 peg plate 975 may comprise chips on top of each of its pegs such that the chip can reach into a well and interact with reagents in the well. A rubber gasket 955 can be placed around the peripheral of the mated plates and between the interface of the two mated plates, thereby providing a better seal.

FIG. 10 shows an illustration of a lengthwise cross-section view of an example incubation device 1000, per some embodiments herein. FIG. 10 shows the incubation device 1000 may comprise a top clamping bracket 1010, a bottom clamping bracket 1020, more than one latch 1030 that clamps the top clamping bracket 1010 and the bottom clamping bracket 1020 together, and more than one rubber foot 1040 on the bottom face of the bottom clamping bracket 1020. Inside the incubation chamber is a pair of mated plates: 384 well pate 1065 and 384 peg plate 1075. Once clamped together by the top and bottom clamping brackets, the 384 well pate 1065 and the 384 peg plate 1075 can be securely fixed inside the incubation chamber residing between the two clamped brackets. The 384 peg plate 1075 may comprise chips on top of each of its pegs such that the chip can reach into a well and interact with reagents in the well. A rubber gasket 1055 can be placed around the peripheral of the mated plates and between the interface of the two mated plates, thereby providing a better seal.

FIG. 11 shows an illustration of another lengthwise cross-section partial view of an example incubation device 1100, per some embodiments herein. In this view. Some of the parts are removed for clarity. FIG. 11 shows the incubation device 1100 may comprise a top clamping bracket 1110, a bottom clamping bracket 1120, more than one latch 1130 that clamps the top clamping bracket 1110 and the bottom clamping bracket 1120 together, and more than one rubber foot 1140 on the bottom face of the bottom clamping bracket 1120. Inside the incubation chamber is a pair of mated plates: 384 well pate 1165 and 384 peg plate 1175. Once clamped together by the top and bottom clamping brackets, the 384 well pate 1165 and the 384 peg plate 1175 can be securely fixed inside the incubation chamber residing between the two clamped brackets. The 384 peg plate 1175 may comprise chips on top of each of its pegs such that the chip can reach into a well and interact with reagents in the well. A rubber gasket 1155 can be placed around the peripheral of the mated plates and between the interface of the two mated plates, thereby providing a better seal.

FIG. 12 shows an illustration of a perspective view of an example peg plate 1200, per some embodiments herein. As shown in FIG. 12, the peg plate 1200 comprises a base layer 1210 and a plurality of pegs 1220 on the base layer 1210. For example, there can be about 10, 20, 30, 40, 50, 60, 70, 80, 90, 100, 150, 200, 250, 300, 350, or 400 pegs 1220 on the base layer 1210. There can be about 16, 32, 64, 96, 192, 256, or 384 pegs 1220 on the base layer 1210. The peg 1210 protrudes outward from the top surface of the base layer 1210. The peg pate 1200 can mate with a complementary well plate such that at least a fraction of the pegs 1210 can reach into the wells of the well plate. For example a 384 peg plate can mate with a 384 well plate such that each of the 384 pegs on the 384 peg plate can reach into a well of the 384 well plate. In addition, the height of the peg 1220 can be adjusted such that the height of the peg 1220 is no more than or shorter than the depth of the well on the well plate. The thickness or shape of the peg 1220 can be adjusted such that when the peg 1220 mates with a well on a well plate, there is still room left in the well other than the peg 1220. In other words, the volume of the peg 1220 is smaller than the volume of the well it mates with on the well plate. Therefore, when the peg 1220 mated with a well, the solution in the well can remain in the well and interact with the peg or other components attached to the peg.

FIG. 13 shows an illustration of a perspective view of an example peg plate 1300 with chips attached to the top of the pegs, per some embodiments herein. As shown in FIG. 13, the peg plate 1300 comprises a base layer 1310, a plurality of pegs 1320 on the base layer 1310, each of the peg 1320 comprises a chip 1330 on the top surface of the peg 1320. For example, there can be about 10, 20, 30, 40, 50, 60, 70, 80, 90, 100, 150, 200, 250, 300, 350, or 400 pegs 1320 on the base layer 1310. There can be about 16, 32, 64, 96, 192, 256, or 384 pegs 1320 on the base layer 1310. The peg 1310 protrudes outward from the top surface of the base layer 1310. The peg pate 1300 can mate with a complementary well plate such that at least a fraction of the pegs 1310 can reach into the wells of the well plate. For example a 384 peg plate can mate with a 384 well plate such that each of the 384 pegs on the 384 peg plate can reach into a well of the 384 well plate. In addition, the height of the peg 1320 can be adjusted such that the height of the peg 1320 is no more than or shorter than the depth of the well on the well plate. The thickness or shape of the peg 1320 can be adjusted such that when the peg 1320 mates with a well on a well plate, there is still room left in the well other than the peg 1320. In other words, the volume of the peg 1320 is smaller than the volume of the well it mates with on the well plate. Therefore, when the peg 1320 mated with a well, the solution in the well can remain in the well and interact with the peg 1320 and the chip 1330 attached to the peg 1320.

In one embodiment, a 384 peg plate with chips on top mates with a 384 well plate. The 384 peg plate, for example, the peg plate 1300 in FIG. 13, can be inserted into a 384 well plate face-down (relative to the configuration shown in FIG. 13), thereby each of the pegs on the peg plate is disposed inside a well on the well plate.

### Terms and Definitions

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

The terminology used herein is for the purpose of describing particular cases only and is not intended to be limiting. As used herein, the singular forms “a”, “an” and “the” can be intended to include the plural forms as well, unless the context clearly indicates otherwise. Furthermore, to the extent that the terms “including”, “includes”, “having”, “has”, “with”, or variants thereof can be used in either the detailed description and/or the claims, such terms can be intended to be inclusive in a manner similar to the term “comprising”.

The term “about” or “approximately” can mean within an acceptable error range for the particular value as determined by one of ordinary skill in the art, which may depend in part on how the value is measured or determined, i.e., the limitations of the measurement system. For example, the term “about” as used herein indicates the value of a given quantity varies by +/−10% of the value, or optionally +/−5% of the value, or in some embodiments, by +/−1% of the value so described. Alternatively, “about” can mean a range of up to 20%, up to 10%, up to 5%, or up to 1% of a given value. Alternatively, particularly with respect to biological systems or processes, the term can mean within an order of magnitude, within 5-fold, or within 2-fold, of a value. Where particular values may be described in the application and claims, unless otherwise stated the term “about” meaning within an acceptable error range for the particular value should be assumed. Also, where ranges and/or subranges of values are provided, the ranges and/or subranges can include the endpoints of the ranges and/or subranges.

The term “substantially” as used herein can refer to a value approaching 100% of a given value. For example, an active agent that is “substantially localized” in an organ can indicate that about 90% by weight of an active agent, salt, or metabolite can be present in an organ relative to a total amount of an active agent, salt, or metabolite. In some cases, the term can refer to an amount that can be at least about 90%, 91%, 92%, 93%, 94%, 95%, 96%, 97%, 98%, 99%, 99.9%, or 99.99% of a total amount. In some cases, the term can refer to an amount that can be about 100% of a total amount.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

The term “label” or “detectable label” as used herein generally refers to any moiety or property that is detectable, or allows the detection of an entity which is associated with the label. For example, a nucleotide, oligo- or polynucleotide that comprises a fluorescent label may be detectable. In some cases, a labeled oligo- or polynucleotide permits the detection of a hybridization complex, for example, after a labeled nucleotide has been incorporated by enzymatic means into the hybridization complex of a primer and a template nucleic acid. A label may be attached covalently or non-covalently to a nucleotide, oligo- or polynucleotide. In some cases, a label can, alternatively or in combination: (i) provide a detectable signal; (ii) interact with a second label to modify the detectable signal provided by the second label, e.g., FRET; (iii) stabilize hybridization, e.g., duplex formation; (iv) confer a capture function, e.g., hydrophobic affinity, antibody/antigen, ionic complexation, or (v) change a physical property, such as electrophoretic mobility, hydrophobicity, hydrophilicity, solubility, or chromatographic behavior. Labels may vary widely in their structures and their mechanisms of action. Examples of labels may include, but are not limited to, fluorescent labels, non-fluorescent labels, colorimetric labels, chemiluminescent labels, bioluminescent labels, radioactive labels, mass-modifying groups, antibodies, antigens, biotin, haptens, enzymes (including, e.g., peroxidase, phosphatase, etc.), and the like. Fluorescent labels may include dyes of the fluorescein family, dyes of the rhodamine family, dyes of the cyanine family, or a coumarine, an oxazine, a boradiazaindacene or any derivative thereof. Dyes of the fluorescein family include, e.g., FAM, HEX, TET, JOE, NAN and ZOE. Dyes of the rhodamine family include, e.g., Texas Red, ROX, R110, R6G, and TAMRA. FAM, HEX, TET, JOE, NAN, ZOE, ROX, R110, R6G, and TAMRA are commercially available from, e.g., Perkin-Elmer, Inc. (Wellesley, Mass., USA), Texas Red is commercially available from, e.g., Thermo Fisher Scientific, Inc. (Grand Island, N.Y., USA). Dyes of the cyanine family include, e.g., CY2, CY3, CY5, CY5.5 and CY7, and are commercially available from, e.g., GE Healthcare Life Sciences (Piscataway, N.J., USA).

The term “different detectable label” or “differently labeled” as used herein generally refers to the detectable label being a different chemical entity or being differentiated among the different bases to which the labels are attached to.

The term “peg plate” as used herein generally refers to a mating plate with respect to a well plate. The peg plate can comprise a base layer and a plurality of pegs on the base layer. The peg plate can mate with a corresponding well plate such that each peg on the peg plate can reach into a well on the well plate. Generally speaking, the height of the peg is no more than or shorter than the depth of the well it mates with. The peg plate can further comprise a plurality of chips, each chip of the plurality of chips is attached to a selected peg of the plurality of pegs. The chip can be attached to the tip of the peg, i.e., the top surface of the peg away from the base layer.

As used herein, the solid substrate used can be biological, non-biological, organic, inorganic, or a combination of any of these. The substrate can exist as one or more particles, strands, precipitates, gels, sheets, tubing, spheres, containers, capillaries, pads, slices, films, plates, slides, or semiconductor integrated chips, for example. The solid substrate can be flat or can take on alternative surface configurations. For example, the solid substrate can contain raised or depressed regions on which synthesis or deposition takes place. In some examples, the solid substrate can be chosen to provide appropriate light-absorbing characteristics. For example, the substrate can be a polymerized Langmuir Blodgett film, functionalized glass (e.g., controlled pore glass), silica, titanium oxide, aluminum oxide, indium tin oxide (ITO), Si, Ge, GaAs, GaP, SiO2, SiN4, modified silicon, the top dielectric layer of a semiconductor integrated circuit (IC) chip, or any one of a variety of gels or polymers such as (poly)tetrafluoroethylene, (poly)vinylidenedifluoride, polystyrene, polycarbonate, polydimethylsiloxane (PDMS), polymethylmethacrylate (PMMA), polycyclicolefins, or combinations thereof.

Solid substrates can comprise polymer coatings or gels, such as a polyacrylamide gel or a PDMS gel. Gels and coatings can additionally comprise components to modify their physicochemical properties, for example, hydrophobicity. For example, a polyacrylamide gel or coating can comprise modified acrylamide monomers in its polymer structure such as ethoxylated acrylamide monomers, phosphorylcholine acrylamide monomers, betaine acrylamide monomers, and combinations thereof.

The term “complementary” as used herein generally refers to a polynucleotide that forms a stable duplex with its “complement,” e.g., under relevant assay conditions. Typically, two polynucleotide sequences that are complementary to each other have mismatches at less than about 20% of the bases, at less than about 10% of the bases, preferably at less than about 5% of the bases, and more preferably have no mismatches.

A “polynucleotide sequence” or “nucleotide sequence” as used herein generally refers to a polymer of nucleotides (an oligonucleotide, a DNA, a nucleic acid, etc.) or a character string representing a nucleotide polymer, depending on context. From any specified polynucleotide sequence, either the given nucleic acid or the complementary polynucleotide sequence (e.g., the complementary nucleic acid) can be determined.

Two polynucleotides “hybridize” when they associate to form a stable duplex, e.g., under relevant assay conditions. Nucleic acids hybridize due to a variety of well characterized physico-chemical forces, such as hydrogen bonding, solvent exclusion, base stacking and the like. An extensive guide to the hybridization of nucleic acids is found in Tijssen (1993) Laboratory Techniques in Biochemistry and Molecular Biology-Hybridization with Nucleic Acid Probes, part I chapter 2, “Overview of principles of hybridization and the strategy of nucleic acid probe assays” (Elsevier, N.Y.), as well as in Ausubel, infra.

The term “polynucleotide” (and the equivalent term “nucleic acid”) encompasses any physical string of monomer units that can be corresponded to a string of nucleotides, including a polymer of nucleotides, e.g., a typical DNA or RNA polymer, peptide nucleic acids (PNAs), modified oligonucleotides, e.g., oligonucleotides comprising nucleotides that are not typical to biological RNA or DNA, such as 2′-O-methylated oligonucleotides, and the like. The nucleotides of the polynucleotide can be deoxyribonucleotides, ribonucleotides or nucleotide analogs, can be natural or non-natural, and can be unsubstituted, unmodified, substituted or modified. The nucleotides can be linked by phosphodiester bonds, or by phosphorothioate linkages, methylphosphonate linkages, boranophosphate linkages, or the like. The polynucleotide can additionally comprise non-nucleotide elements such as labels, quenchers, blocking groups, or the like. The polynucleotide can be, e.g., single-stranded or double-stranded.

The term “oligonucleotide” as used herein generally refers to a nucleotide chain. In some cases, an oligonucleotide is less than 200 residues long, e.g., between 15 and 100 nucleotides long. The oligonucleotide can comprise at least or about 1, 2, 3, 4, 5, 6, 7, 8, 9, 10, 15, 20, 25, 30, 35, 40, 45, or 50 bases. The oligonucleotides can be from about 3 to about 5 bases, from about 1 to about 50 bases, from about 8 to about 12 bases, from about 15 to about 25 bases, from about 25 to about 35 bases, from about 35 to about 45 bases, or from about 45 to about 55 bases. The oligonucleotide (also referred to as “oligo”) can be any type of oligonucleotide (e.g., a primer). Oligonucleotides can comprise natural nucleotides, non-natural nucleotides, or combinations thereof.

### EXAMPLES

The following illustrative examples are representative of embodiments of the software applications, systems, and methods described herein and are not meant to be limiting in any way.

### Example 1

### Protocol for Bubble Reduction

The followings are steps in a protocol for overnight a hybridization process:

1. Load 384 well plate with appropriate hybridization reagents (30 μL/well or as indicated in Hybridization-Ligation Assay Protocol PSP00019, available at Centrillion Technolgies, Inc., Palo Alto, Calif., USA 94303)

2. Insert a 384 chip plate into the well plate by aligning the notched corner and pressing gently until the plates are coupled. Due to plate loading, some bubbles may already be trapped and visible inside the wells.

3. Place the mated pair inside the SORVALL LEGEND RT centrifuge equipped with swing-buckets for standard 384 well plates.
Note: Ensure the rotor is balanced by loading even number of plates, or by placing a dummy 384 plate with equivalent load in the opposite bay.

4. (First Centrifuge step) Set the centrifuge to rotate for 1 min at 3750 rpm. Let the centrifuge come to a complete stop prior to removing the mated plates. At this point, there should be no bubbles in the well plate.

5. Clamp the mated plates:
a. If using the humidity chamber (“lunch box”), clamp the plates using binder clips, and place the mated plates horizontally inside the humidity chamber. Close the lid and place the chamber inside the hybridization oven preset at 48° C.
b. If using the 384 plate clamp, insert the gasket between the well plate and chip plate. Close the 4 latches to seal the plate pair and place the assembly horizontally inside the hybridization oven preset at 48° C.

6. (First Incubation step) After 3 hours of incubation, remove the clamped plates from the hybridization oven. There may be some air bubbles formed inside the wells.

7. Remove the binder clips/plate clamp to recover the mated 384 plate pair. DO NOT separate the 384 chip plate from the 384 well plate.

8. (Second Centrifuge step) Place the mated pair inside the SORVALL LEGEND RT centrifuge again and set the centrifuge to rotate for 2 mins at 3750 rpm. Let the centrifuge come to a complete stop prior to removing the mated plates. At this point, there should be no bubbles in the well plate.

9. (Second Incubation step) Clamp the mated plates (repeating step 3), continue the 48° C. incubation for the remaining time period (18 hours, or as indicated in the assay protocol, Hybridization-Ligation Assay Protocol PSP00019).

### Example 2

### Hybridization of a Hybridization Solution

In one example, per FIG. 4, 10 well plates each holding 384 wells or 96 wells were filled with a hybridization solution and processed per Table 1 below according to general steps of the protocol disclosed in Example 1 unless described otherwise (e.g., with or without the centrifuge step, the incubation time, etc.). The hybridization conditions are: 21 hours total at 48° C. unless stated otherwise. The hybridization solution contains hybridization buffers used for the hybridization reactions, but without DNA, 30 μL volume per well.

TABLE 1
Bubble Suppression Test Results on 384- or 96-well Platform

| | | | | Bubble Count | | | | |
|---|---|---|---|---|---|---|---|---|
| Test | # of | Tween- | 1st | Before 1st | After 1st | After 2nd | After 2nd | Percentage of wells |
| No. | wells | 20 | Centrifuge | Incubation | Incubation | Centrifuge | Incubation | with bubbles |
| 1* | 384 | yes | no | 17 | — | — | 49 | 13% |
| 2 | 96 | yes | yes | 0 | — | — | 17 | 18% |
| 3 | 384 | yes | yes | 0 | 6 | 0 | 8 | 2% |
| 4 | 384 | yes | yes | 0 | 7 | 0 | 1 | 0% |
| 5 | 384 | yes | no | 18 | 34 | 0 | 4 | 1% |
| 6 | 384 | yes | yes | 0 | 2 | 0 | 3 | 1% |
| 7 | 384 | yes | yes | 0 | 3 | 0 | 2 | 1% |
| 8 | 96 | yes | no | 2 | 5 | 0 | 1 | 1% |
| 9 | 96 | yes | yes | 0 | 1 | 0 | 0 | 0% |
| 10 | 96 | no | yes | 0 | 2 | 0 | 1 | 1% |

Notes for tests in Table 1:
Test No. 1: A control experiment without any centrifugation (i.e., no first or second centrifuge steps)
Test No. 2: Only have the first centrifuge step (i.e., having one centrifuge before the incubation)
Test No. 3: The mated plates are placed in a humidity chamber without clamps or binding clips to clamp the two plates.
Test No. 4: Using longer (i.e., 5 minutes) centrifuge time for the second centrifuge step.
Test No. 6: Using shorter (i.e., 1 hour) for the first incubation (e.g., 1 hour).
The first centrifuge step (i.e., centrifuge prior to the 3-hour incubation step, or “Pre-centrifuge”) is about 1-minute long.
The first incubation is about 3-hour long except for Test No. 6.
The second centrifuge step (i.e., centrifuge after the 3-hour incubation step) is about 2-minute long except for Text No. 4.
The second incubation is about 18-hour long.

As seen in Table 1 above, Test No. 1 shows that without any centrifugation, the bubbles can be formed at the start of the incubation step when the two plates are mated. At the end of the 18-hour incubation step more bubbles are formed. Further, comparing Test Nos. 1 and 2, the first centrifuge step removes bubbles at the start of the incubation and at the end of the 18-hour incubation when compared with protocols having no centrifuge step in a head-to-head comparison. Comparing Test Nos. 1 and 5 shows that protocols having a second centrifuge step (i.e., a centrifuge step after 3-hour of incubation) can reduce the amount of bubbles at the end of the 18-hour incubation. Comparing Test Nos. 2 and 9 shows that the addition of the second centrifuge step additionally reduced the number of bubbles in the hybridization solution. As such, the method of centrifuging, incubating, centrifuging, and incubating the hybridization solution with the Tween-20 surfactant yields less than 2 percent of wells having bubbles. In some embodiments, the method of centrifuging, incubating, centrifuging, and incubating, the hybridization solution with the Tween-20 surfactant yields less than 1 percent of wells having bubbles.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure.

What is claimed is:

1. A hybridization method with reduced bubble formation, the method comprising:
(a) centrifuging a hybridization solution disposed in a plurality of wells of a well plate at a first speed for a first centrifuging time period;
(b) incubating the hybridization solution at a first temperature for a first incubation time period;
(c) centrifuging the hybridization solution at a second speed for second centrifuging time period; and
(d) incubating the hybridization solution at a second temperature for a second incubation time period;
wherein at most 1% of the plurality of wells of the well plate have a bubble within the hybridization solution.

2. The method of claim 1, wherein the first speed is from about 1,875 rpm to about 7,500 rpm, and wherein the first centrifuging time period is from about 0.5 minutes to about 2 minutes.

3. The method of claim 1, wherein the first temperature is from about 24° C. to about 92° C., and wherein the first incubation time is from about 1 hour to about 6 hours.

4. The method of claim 1, wherein the second speed is about 1,875 rpm to about 7,500 rpm, and wherein the second centrifuging time period is about 1 minute to about 10 minutes.

5. The method of claim 1, wherein the second temperature is about 24° C. to about 92° C., and wherein the second incubation time is about 9 hours to about 36 hours.

6. The method of claim 1, wherein the first speed is equal to the second speed.

7. The method of claim 1, wherein the first centrifuging time period is equal to the second centrifuging time period.

8. The method of claim 1, wherein the first temperature is equal to the second temperature.

9. The method of claim 1, wherein the first incubation time is equal to the second incubation time.

10. The method of claim 1, wherein the hybridization solution comprises dimethyl sulfoxide (DMSO), polyoxyethylene (20) sorbitan monolaurate (Tween-20), 2-amino-2-(hydroxymethyl) propane-1,3-diol and buffer (Tris) and ethylenediaminetetraacetic acid (EDTA) buffer (Tris-EDTA buffer), tetramethylammonium chloride (TMAC), and water.

11. The method of claim 10, wherein the Tris-EDTA buffer has a pH of about 8.

12. The method of claim 10, wherein a volume percentage of the DMSO in the hybridization solution is from about 5% to about 20%

13. The method of claim 10, wherein a volume percentage of the Tween-20 in the hybridization solution is from about 0.025% to about 0.1%.

14. The method of claim 10, wherein a concentration of the Tris-EDTA buffer in the hybridization solution is from about 0.02 M to about 0.1 M for Tris, and from about 0.5 mM to about 2 mM for EDTA.

15. The method of claim 10, wherein a molar ratio of Tris:EDTA in the hybridization solution is from about 20 to about 100.

16. The method of claim 10, wherein a concentration of the TMAC in the hybridization solution is from about 2 M to about 4M.

17. The method of claim 10, wherein a volume percentage of the water in the hybridization solution is from about 12% to about 49%.

18. The method of claim 10, wherein the water is nuclease-free water.

19. The method of claim 1, wherein a volume of the hybridization solution disposed within each of the plurality of wells is from about 15 μL to about 60 μL.

20. A hybridization device comprising:
a) a top clamping bracket;
b) a bottom clamping bracket;
c) one or more latches configured to clamp the top clamping bracket and the bottom clamping bracket together;
d) a reaction chamber defined by the clamped top clamping bracket and the bottom clamping bracket;
e) a well plate disposed within the reaction chamber, the well plate comprising a plurality of wells; and
f) a peg plate disposed within the reaction chamber, the peg plate comprising a plurality of pegs;
wherein the peg plate mates with the well plate, thereby each peg of the plurality of pegs mates with a selected well of the plurality of wells.
